(19) United States
(12) Patent Application Publication
(10) Pub. No.: US 2010/0319096 A1
(43) Pub. Date: Dec. 23, 2010

(54) METHODS AND DEVICES FOR REDUCING ELBOW PAIN AND ARM FATIGUE FOR POKER PLAYERS WHEN PLAYING POKER

(76) Inventors: Michael A. SCOTT, Rensselaer, NY (US); Henry G. HIGGITT, Rensselaer, NY (US)

Correspondence Address: HESLIN ROTHENBERG FARLEY & MESITI PC, 5 COLUMBIA CIRCLE, ALBANY, NY, 12203, US

(21) Appl. No.: 12/489,125

(22) Filed: Jun. 22, 2009

(51) Int. Cl. A41D 13/08; A61F 7/00; A63B 71/00

(52) U.S. Cl. 2/16; 607/108; 607/114; 273/459

(57) ABSTRACT

A method includes in one embodiment wearing a first resilient sleeve having a first elbow pad on a first arm of the poker player, positioning the elbow pad between a surface of a poker table and the poker player's elbow of the first arm, and compressing a portion of the first resilient elbow pad between a surface of a poker table and the poker player's first elbow of the first arm to resiliently support the weight of the poker player's first arm on the first elbow pad during play of a plurality of poker games. The method may further include positioning a hot pack or a cold pack in a pocket attached to the sleeve on the first arm during play of the plurality of poker games.

### FIELD OF THE INVENTION

This invention relates generally to playing the game of poker, and more particularly to methods and devices for reducing elbow and arm fatigue for poker players when playing poker.

### BACKGROUND OF THE INVENTION

Poker players often experience and complain about sore elbows and arms from the repetitive motion of placing and resting their elbows and forearms on a poker table or a railing of a poker table for extended amounts of time when playing poker. Further, poker players often bruise the skin under their elbows from the repetitive motion of sliding their elbows and forearms on the poker table or railing when reaching for their cards when playing poker.

There is a need for methods and devices for reducing elbow and arm fatigue and alleviating discomfort for poker players when playing poker.

### SUMMARY OF THE INVENTION

In a first aspect, the present invention provides a method for reducing elbow and arm fatigue of a poker player during play of a plurality of poker games. The method includes providing a first resilient elbow padding having a first thickness, releaseably maintaining the first resilient elbow padding over at least one of the poker player's outer clothing on a first arm and the poker player's bare first arm without clothing so that a portion of the first resilient elbow padding is disposed over the poker player's elbow of the first arm during play of the plurality of poker games, positioning the first resilient elbow padding between a first surface of a poker table and the poker player's elbow of the first arm during play of the plurality of poker games, and compressing a portion of the first resilient elbow padding to a second thickness less than the first thickness between the first surface of the poker table and the poker player's elbow of the first arm to distribute the weight of the poker player's first arm on the poker player's elbow of the first arm over a greater area during play of the plurality of poker games compared to an area for distributing the weight of the poker player's first arm on the elbow of the first arm disposed directly on the poker table during play of the plurality of poker games.

In a second aspect, the present invention provides a method for reducing elbow and arm fatigue of a poker player during play of a plurality of poker games. The method includes wearing a first sleeve having a first elbow pad on a first arm of the poker player, the first sleeve having a terminating open end disposed between a wrist and an elbow of the poker player and a terminating open end disposed between a shoulder and the elbow of the poker player, and the first elbow pad being disposed over the elbow of the first arm, positioning the first elbow pad between a surface of a poker table and the poker player's elbow of the first arm, and compressing a portion of the first elbow pad between the surface of the poker table and the poker player's elbow of the first arm to support the weight of the poker player's first arm on the first elbow pad during play of the plurality of poker games.

In a third aspect, the methods may further comprise reducing elbow and arm fatigue of both right and left arms of a poker player during play of a plurality of poker games. Further, in a fourth aspect, at least one of a hot pack and a cold pack may be positioned on the poker player's first arm during play of the plurality of poker games, and at least one of a hot pack and a cold pack may be positioned on the poker player's second arm during play of the plurality of poker games.

In a fifth aspect, the present invention provides a device for reducing elbow and arm fatigue of a poker player during play of a plurality of poker games. The device includes means for releasably covering a portion of at least one of the poker player's outer clothing on a first arm and the poker player's bare first arm without clothing so that a portion of the covering means is disposed over the poker player's elbow of the first arm during play of the plurality of poker games, said covering means having an upper terminating end positionable below the shoulder of the poker player and a lower terminating end positionable above the wrist of the poker player, and means, attached to said covering means and positionable over the elbow of the first arm of the poker player, for distributing the weight of the poker player's first arm on the poker player's first elbow over a greater area of the poker table during play of a plurality of poker games compared to an area for distributing the weight of the poker player's first arm on the first elbow disposed directly on the poker table during play of the plurality of poker games.

In a sixth aspect, the present invention provides a kit for reducing elbow and arm fatigue of a poker player during play of a poker game. The kit includes a plurality of the above-noted devices, a first one of the plurality of devices positionable on a right arm of the poker player, and a second one of the plurality of devices positionable on a left arm of the poker player. The kit may further include at least one of heating and cooling means such as at least one of a hot pack and a cold pack.

### BRIEF DESCRIPTION OF THE DRAWINGS

The subject matter which is regarded as the invention is particularly pointed out and distinctly claimed in the concluding portion of the specification. The invention, however, may best be understood by reference to the following detailed description of various embodiments and the accompanying drawings in which:

FIG. 1 is a perspective view of a first embodiment of a device having an elbow pad for reducing elbow and arm fatigue for poker players when playing poker in accordance with the present invention;

FIG. 2 is a perspective view of a second embodiment of a device having an elbow pad for reducing elbow and arm fatigue for poker players when playing poker in accordance with the present invention;

FIG. 3 is a perspective view of a third embodiment of device having an elbow pad for reducing elbow and arm fatigue for poker players when playing poker in accordance with the present invention;

FIG. 4 is a plan view of the device of FIG. 3;

FIG. 5 is a perspective view of a fourth embodiment of a device having a pocket for a hot pack or a cold pack for reducing arm fatigue for poker players when playing poker in accordance with the present invention;

FIG. 6 is a perspective view of a fifth embodiment of a device having a pocket for a hot pack or a cold pack for reducing arm fatigue for poker players when playing poker in accordance with the present invention;

FIG. 7 is a perspective view of a sixth embodiment of device having a pocket for a hot pack or a cold pack for reducing arm fatigue for poker players when playing poker in accordance with the present invention;

FIG. 8 is a plan view of device of FIG. 7;

FIG. 9 is a perspective view of a seventh embodiment of a device having an elbow pad and a pocket for a hot pack or a cold pack for reducing elbow and arm fatigue for poker players when playing poker in accordance with the present invention;

FIG. 10 is a perspective view of an eighth embodiment of a device having an elbow pad and a pocket for a hot pack or a cold pack for reducing elbow and arm fatigue for poker players when playing poker in accordance with the present invention;

FIG. 11 is a perspective view of a ninth embodiment of device having an elbow pad and a pocket for a hot pack or a cold pack for reducing elbow and arm fatigue for poker players when playing poker in accordance with the present invention;

FIG. 12 is a plan view of the device of FIG. 11;

FIG. 13 is a perspective view of a tenth embodiment of a device having an elbow pad, a pocket for a hot pack or a cold pack, and a pocket for an electronic device, for reducing elbow and arm fatigue for poker players when playing poker in accordance with the present invention;

FIG. 14 is a perspective view of an eleventh embodiment of a device having an elbow pad, a pocket for a hot pack or a cold pack, and a pocket for an electronic device, for reducing elbow and arm fatigue for poker players when playing poker in accordance with the present invention;

FIG. 15 is a perspective view of a twelfth embodiment of device having an elbow pad, a pocket for a hot pack or a cold pack, and a pocket for an electronic device, for reducing elbow and arm fatigue for poker players when playing poker in accordance with the present invention;

FIG. 16 is a plan view of the device of FIG. 15;

FIG. 17 is a cross-sectional view of one embodiment of the elbow portion of the various devices in accordance with the present invention;

FIG. 18 is a cross-sectional view of another embodiment of the elbow portion of the various devices in accordance with the present invention;

FIG. 19 is a cross-sectional view of another embodiment of the elbow portion of the devices for playing poker at a poker table;

FIG. 20 is a cross-sectional view of the elbow portion of the device of FIG. 19 worn by a poker player and resting his elbow on a poker table when playing poker;

FIG. 21 is a cross-sectional view of the elbow of the poker player not wearing the device and resting his elbow directly on a poker table when playing poker;

FIG. 22 is a diagrammatic illustration of the forces exerted on an elbow of the poker player when wearing the device as shown in FIG. 20 when playing poker at a poker table;

FIG. 23 is a diagrammatic illustration of the forces exerted on an elbow of the poker player when not wearing the devices of the present invention as shown in FIG. 21 when playing poker at a poker table;

FIG. 24 is a cross-sectional view through an open pocket of the devices of the present invention;

FIG. 25 is a flowchart of a first embodiment of a method for reducing elbow and arm fatigue of a poker player during play of a plurality of poker games in accordance with the present invention; and

FIG. 26 is a flowchart of another embodiment of a method for reducing elbow and arm fatigue of a poker player during play of a plurality of poker games in accordance with the present invention.

### DETAILED DESCRIPTION OF THE INVENTION

The present invention is directed to devices and methods for reducing elbow and arm fatigue of poker players when the poker players play a plurality of poker hands, rounds, or games, and in particular, play a plurality of poker hands, rounds, or games during indoor poker tournaments which can last many hours and extend over many days. For example, the devices in accordance with the present invention may include a sleeve which is worn over an arm of the poker player, or a pair of sleeves which can be worn over both arms during play of the plurality of poker games, to reduce the likelihood of the poker player's arm becoming tender and sensitive due to generally constant pressure and frictional contact with the surface of the poker table. The devices may be operable to relieve pressure points such as reduce pressure on the elbow and forearm of the poker player. In addition, the present invention, by reducing elbow and arm fatigue when worn by poker players during play of a plurality of poker games, may result in the poker players being less distracted, more relaxed, and better able to concentrate when playing the plurality of poker games.

The devices may include a sleeve and a resilient elbow pad which is compressible between a surface of a poker table and the poker player's elbow, e.g., the bony prominence at the very tip of the elbow, to support the weight of the poker player's arm and reduce the force exerted on the corner or lowermost portion of the player's elbow during play of a plurality of poker games compared to the force exerted on the lowermost portion of the poker player's elbow for supporting the player's elbow directly on the poker table or on the clothing worn on the arm of the poker player. For example, the resilient elbow pad may be compressible to distribute the weight of the poker player's arm on the elbow over a greater area during play of the plurality of poker games compared to an area for distributing the weight of the poker player's first arm on the elbow disposed directly on the poker table. The device desirably distributes the weight of the poker player's arm whether the forearm is rested on the poker table, the forearm of the poker player's arm elevated above the poker table, and whether the forearm of the poker player's arm elevated above the poker table with the elbows spaced-apart and the hands held adjacent to each other so that the arms are angled toward each other.

The sleeve may include means such as an open pocket for receiving a hot pack or a cold pack and for reducing arm fatigue and/or providing therapeutic treatment to the poker player's arm. The sleeve may further include means such as an open pocket for receiving and holding at least one of an electronic device such as an MP3 player, a cell phone, keys, and glasses.

While the present invention is suitable for poker players, it will be appreciated that the devices and methods of the present invention for reducing elbow and arm fatigue may be suitable for use by workers for resting their elbows and forearms on chair arms or other surfaces when working on a keyboard and/or viewing a computer or monitor, truck drivers and other drivers when driving and resting their elbows and forearms on seat arm rests or other arm rests, and others when repetitively placing and/or sliding their elbows or forearms on a hard surface.

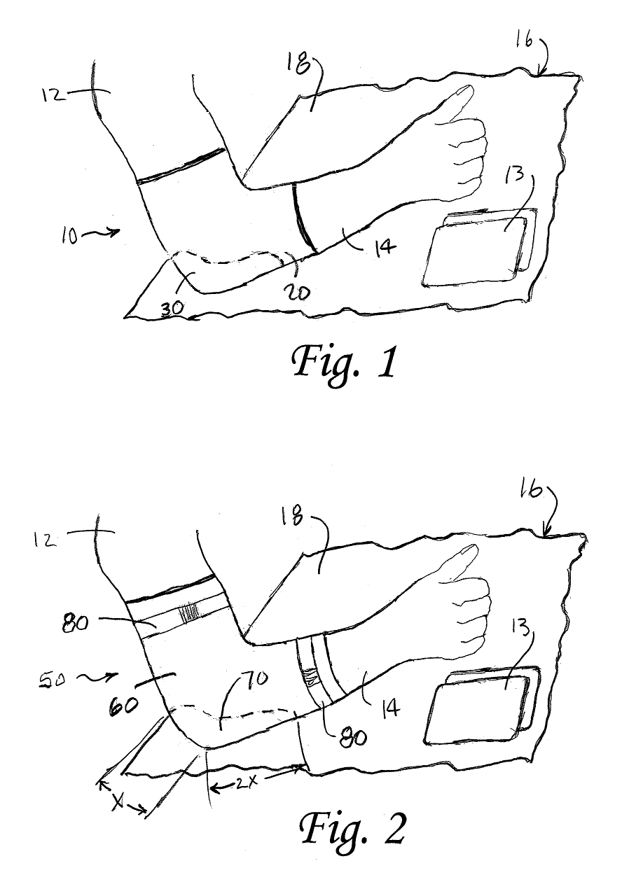

FIG. 1 illustrates a first embodiment of a device 10 in accordance with the present invention for reducing elbow and arm fatigue for a poker player 12 when playing a plurality of poker hands or games involving a plurality of cards 13. In this embodiment, device 10 may include a sleeve 20 positionable over an arm 14 of poker player 12. Sleeve 20 may extend and terminate between the poker player's upper arm and elbow, and elbow and forearm. Sleeve 20 may include resilient means 30, attached to sleeve 20, for resiliently supporting an elbow of the poker player on a poker table 16, for example, on a edge or rail 18 of poker table 16, during play of the plurality of poker hands or games. The sleeve may be fabricated from a resilient or stretchable fabric or material, a non-stretchable fabric or material, or combinations thereof as further described below.

FIG. 2 illustrates a second embodiment of a device 50 in accordance with the present invention for reducing elbow and arm fatigue for a poker player 12 when playing a plurality of poker hands or games involving a plurality of cards 13. In this embodiment, device 50 may include a sleeve 60 positionable over an arm 14 of poker player 12. Sleeve 60 may extend and terminate between the poker player's upper arm and elbow, and elbow and forearm. Sleeve 60 may include resilient means 70, attached to sleeve 60, for resiliently supporting an elbow of the poker player on a poker table 16, for example, on a edge or rail 18 of the poker table, during play of the plurality of poker hands or games, and at least one strap 80 for holding the sleeve on the poker player's arm. The sleeve may be fabricated from a resilient or stretchable fabric or material, a non-stretchable fabric or material, or combinations thereof as further described below. The sleeve may also configured slightly larger in size than the poker player's arm.

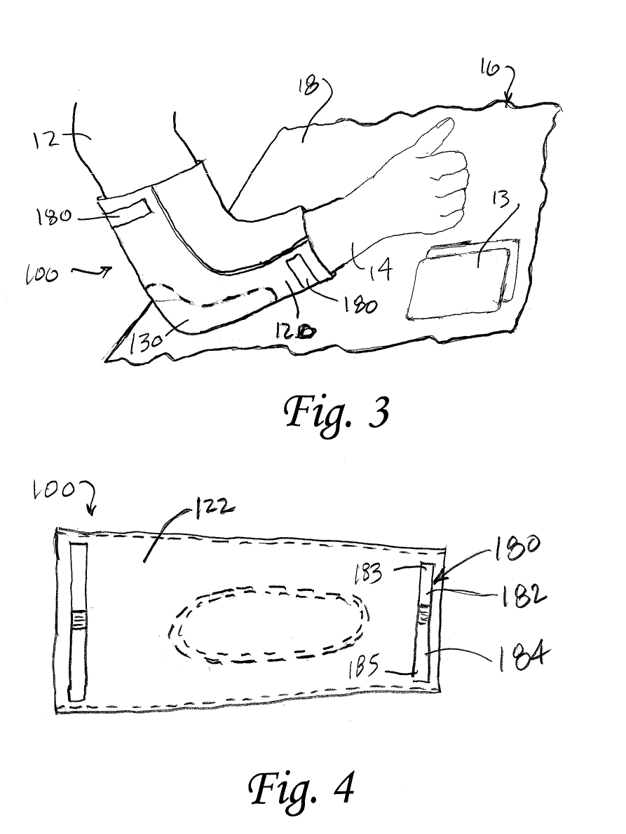

FIG. 3 illustrates a third embodiment of a device 100 in accordance with the present invention for reducing elbow and arm fatigue for a poker player 12 when playing a plurality of poker hands or games employing a plurality of cards 13. In this embodiment, device 100 may include a generally loosely fit sleeve 120 positionable over an arm 14 of poker player 12. Sleeve 120 may extend and terminate between the poker player's upper arm and elbow, and elbow and forearm. Sleeve 120 may include resilient means 130, attached to sleeve 120, for resiliently supporting an elbow of the poker player on a poker table 16, and at least one strap 180 for holding the sleeve on the poker player's arm. When the device is worn by the poker player, the resilient means resiliently supports the elbow of the poker player on a poker table 16, and for example, on a edge or rail 18 of poker table 16, during play of the plurality of poker hands or games. As shown in FIG. 4, device 100 may be formed from a plurality of generally rectangular-shaped or tapering layers 122 (only one of which is shown in FIG. 4), and sewn together along the longitudinally-extending edges of the plurality of layers. The sleeve may be fabricated from a resilient or stretchable fabric or material, a non-stretchable fabric or material, or combinations thereof as further described below.

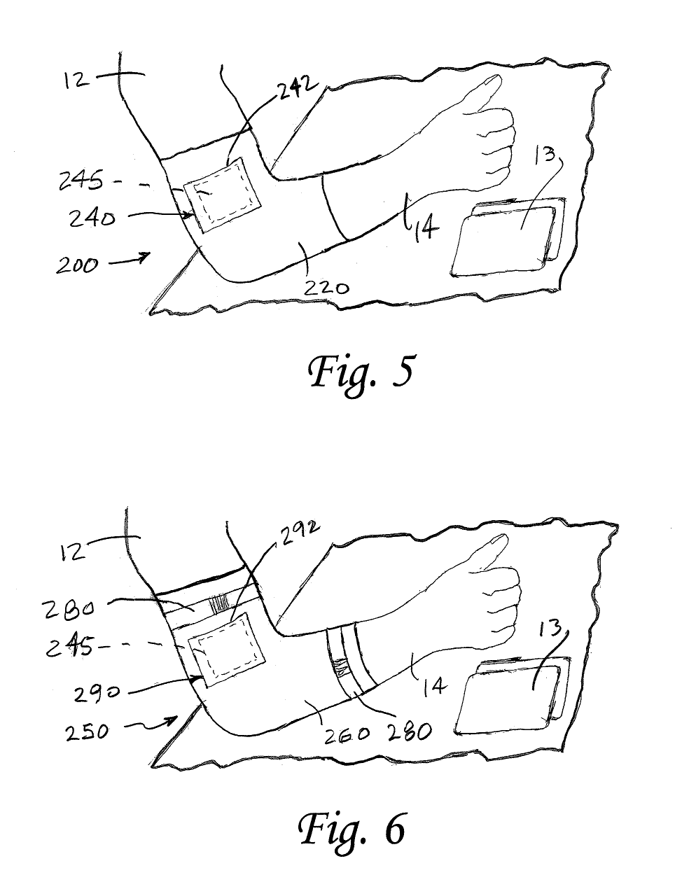

FIG. 5 illustrates a fourth embodiment of a device 200 in accordance with the present invention for reducing arm fatigue for a poker player 12 when playing a plurality of poker hands or games employing a plurality of cards 13. In this embodiment, device 200 may include a sleeve 220 positionable over an arm 14 of poker player 12. Sleeve 220 may extend and terminate between the poker player's upper arm and elbow, and elbow and forearm. Sleeve 220 may include means 240, positionable over a portion of the player's arm, for receiving at least one of a hot pack and a cold pack 245 for reducing arm fatigue of the poker player during play of the poker game. Means 240 may include an open pocket 242 having an open end along the top thereof. The sleeve may be fabricated from a resilient or stretchable fabric or material, a non-stretchable fabric or material, or combinations thereof as further described below.

FIG. 6 illustrates a fifth embodiment of a device 250 in accordance with the present invention for reducing arm fatigue for a poker player 12 when playing a plurality of poker hands or games using a plurality of cards 13. In this embodiment, device 250 may include a sleeve 260 positionable over an arm 14 of poker player 12. Sleeve 260 may extend and terminate between the poker player's upper arm and elbow, and elbow and forearm. Sleeve 260 may include means 290, positionable over a portion of the player's arm, for receiving at least one of a hot pack and a cold pack 245 for reducing arm fatigue of the poker player during play of the poker game, and at least one strap 280 for holding the sleeve on the poker player's arm. Means 290 may include an open pocket 292 having an open end along the top thereof. The sleeve may be fabricated from a resilient or stretchable fabric or material, a non-stretchable fabric or material, or combinations thereof as further described below. The sleeve may also configured slightly larger in size than the poker player's arm.

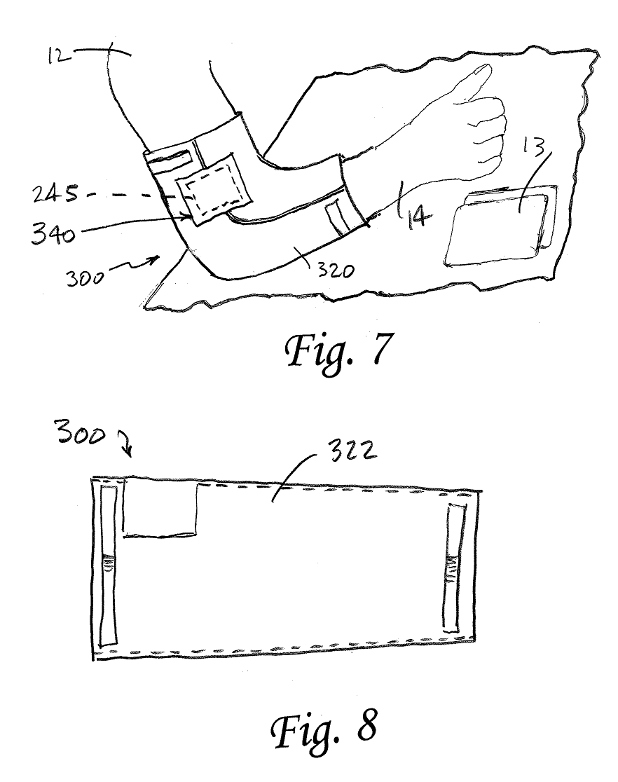

FIG. 7 illustrates a sixth embodiment of a device 300 in accordance with the present invention for reducing arm fatigue for a poker player 12 when playing a plurality of poker hands or games employing a plurality of cards 13. In this embodiment, device 300 may include a generally loosely fit sleeve 320 positionable over an arm 14 of poker player 12. Sleeve 320 may extend and terminate between the poker player's upper arm and elbow, and elbow and forearm. Sleeve 320 may include means 340, positionable over a portion of the player's arm, for receiving at least one of a hot pack and a cold pack 245 for reducing arm fatigue of the poker player during play of the plurality of poker games. As shown in FIG. 8, device 300 may be formed from a plurality of generally rectangular-shaped or tapering layers 322 (only one of which is shown in FIG. 8), and sewn together along the longitudinally-extending edges of the plurality of layers. The sleeve may be fabricated from a resilient or stretchable fabric or material, a non-stretchable fabric or material, or combinations thereof as further described below. The sleeve may also be configured slightly larger in size than the poker player's arm.

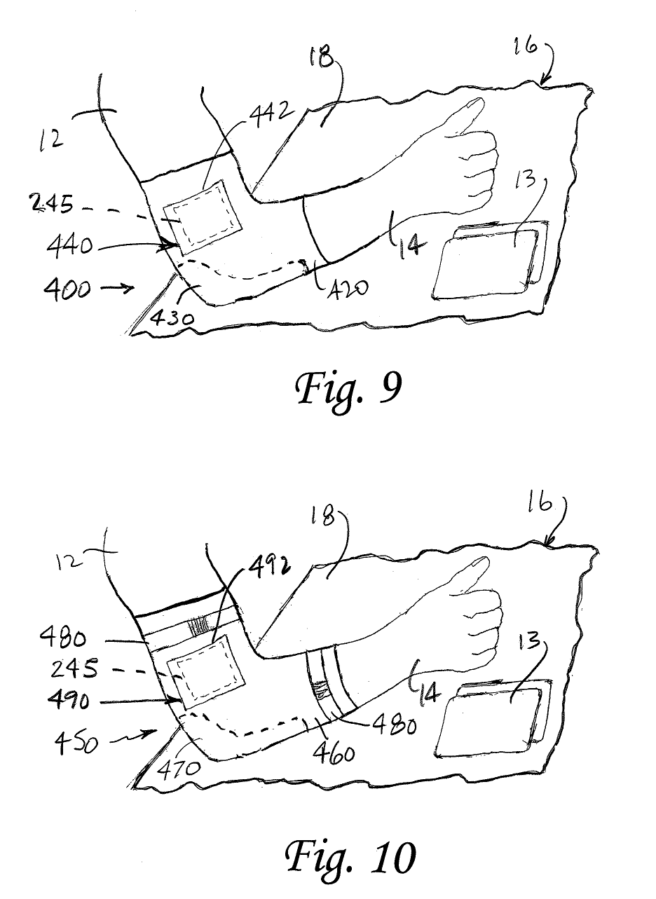

FIG. 9 illustrates a seventh embodiment of a device 400 in accordance with the present invention for reducing elbow and arm fatigue for a poker player 12 when playing a plurality of poker hands or games using a plurality of cards 13. In this embodiment, device 400 may include a sleeve 420 positionable over an arm 14 of poker player 12. Sleeve 420 may extend and terminate between the poker player's upper arm and elbow, and elbow and forearm. Sleeve 420 may include resilient means 430, attached to sleeve 420, for resiliently supporting an elbow of the poker player on a poker table 16, for example, on a edge or rail 18 of the poker table, during play of the plurality of poker hands or games. Sleeve 420 may also include means 440, positionable over a portion of the player's arm, for receiving at least one of a hot pack and a cold pack 245 for reducing arm fatigue of the poker player during play of the plurality of poker hands or games. For example, means 440 may include an open pocket 442. The sleeve may be fabricated from a resilient or stretchable fabric or material, a non-stretchable fabric or material, or combinations thereof as further described below.

FIG. 10 illustrates an eighth embodiment of a device 450 in accordance with the present invention for reducing elbow and arm fatigue for a poker player 12 when playing a plurality of poker hands or games. In this embodiment, device 450 may include a sleeve 460 positionable over an arm 14 of poker player 12. Sleeve 460 may extend and terminate between the poker player's upper arm and elbow, and elbow and forearm. Sleeve 460 may include resilient means 470, attached to sleeve 460, for resiliently supporting an elbow of the poker player on a poker table 16, for example, on a edge or rail 18 of poker table 16, during play of the plurality of poker games, and at least one strap 480 for securing the sleeve to the player's arm. Sleeve 460 may also include means 490, positionable over a portion of the player's arm, for receiving at least one of a hot pack and a cold pack 245 for reducing arm fatigue of the poker player during play of the plurality of poker hands or games. For example, means 490 may include an open pocket 492. The sleeve may be fabricated from a resilient or stretchable fabric or material, a non-stretchable fabric or material, or combinations thereof as further described below. The sleeve may also configured slightly larger in size than the poker player's arm.

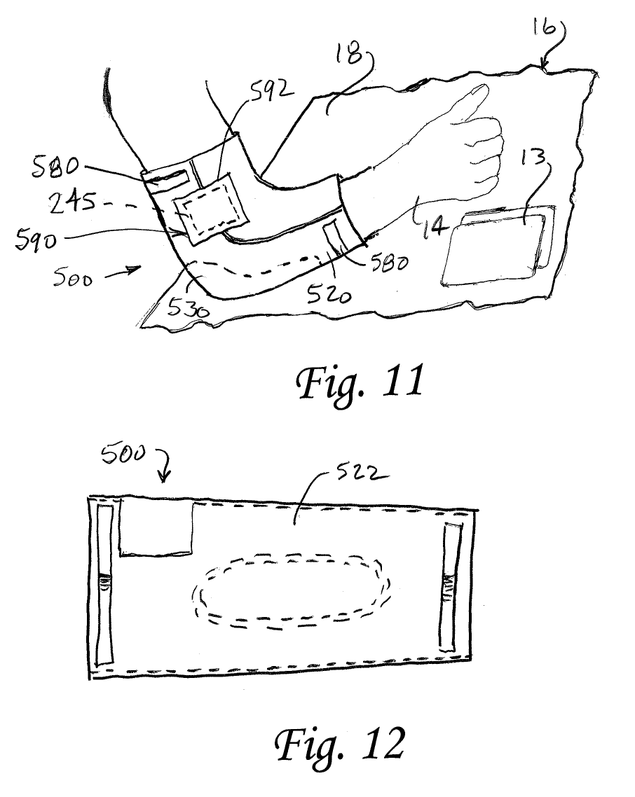

FIG. 11 illustrates a ninth embodiment of a device 500 in accordance with the present invention for reducing elbow and arm fatigue for a poker player 12 when playing a plurality of poker hands or games. In this embodiment, device 500 may include a generally loosely fit sleeve 520 positionable over an arm 14 of poker player 12. Sleeve 520 may extend and terminate between the poker player's upper arm and elbow, and elbow and forearm. Sleeve 520 may include resilient means 530, attached to sleeve 520, for resiliently supporting an elbow of the poker player on a poker table 16, and at least one strap 580 for securing the sleeve to the player's arm. When the device is worn by the poker player, the resilient means resiliently supports the elbow of the poker player on poker table 16, and for example, on an edge or rail 18 of poker table 16, during play of the poker games. Sleeve 520 may also include means 590, positionable over a portion of the player's arm, for receiving at least one of a hot pack and a cold pack 245 for reducing arm fatigue of the poker player during play of the poker game. For example, means 590 may include an open pocket 592. As shown in FIG. 12, device 500 may be formed from a plurality of generally rectangular-shaped or tapering layers 522 (only one of which is shown in FIG. 12), and sewn together along the longitudinally-extending edges of the plurality of layers.

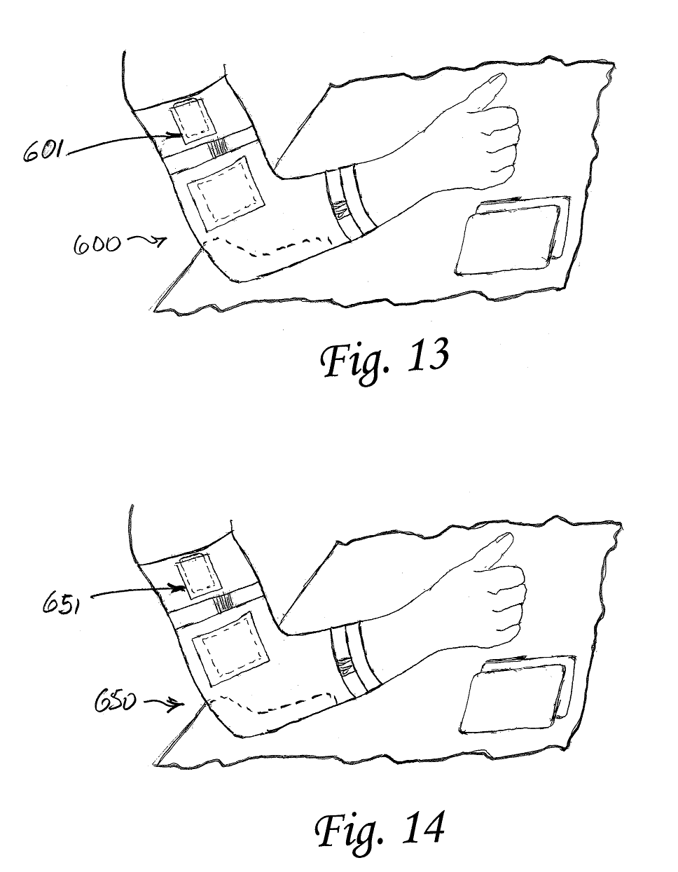

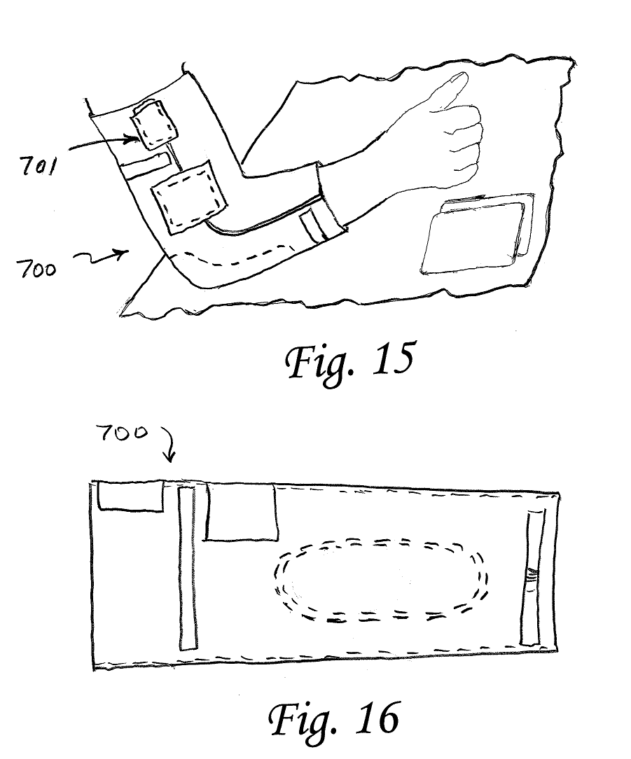

FIGS. 13-16 illustrate further embodiments of devices 600, 650, and 700, of the present invention for reducing elbow and arm fatigue for a poker player when playing a plurality of poker hands or games. Devices 600, 650, and 700 are essentially the same as the embodiments shown in FIGS. 9-12, with the exception that of the sleeve may include additional means 601, 651, and 701, positionable over a third portion of the player's first arm, for receiving and holding at least one of an electronic device, an MP3 player, keys, and glasses during play of the plurality of poker hands or games. For example, the means may include an open pocket. The open pocket may be sized smaller than the means for receiving at least one of the hot pack and the cold pack for reducing arm fatigue of the poker player during play of the poker game. Further embodiments may include a sleeve having an elbow pad and a pocket for receiving and holding at least one of an electronic device, an MP3 player, keys, and glasses during play of the plurality of poker hands or games. Other embodiments may include a sleeve having a pocket for receiving a hot pack or cold pack, and a pocket for receiving and holding at least one of an electronic device, an MP3 player, keys, and glasses during play of the plurality of poker hands or games.

The various above-described embodiments may include one or more of the following features. For example, the sleeve may be contoured to the shape of a poker player's right arm or left arm and formed from an elastic material such as an elastic material such as a LYCRA material, a SPANDEX material, and other suitable 1-way, two-way, and all-way stretch materials and the like. Alternatively, the sleeve may be formed from a generally non-stretch material such as a generally thick non-stretch fabric or cushioning fabric such as fleece, cotton polyester and/or be loosely fit or snugly fit over a poker player's right arm or left arm. For example, the sleeve may be formed from a polar fleece (also known as microfleece or micrafleece) a soft napped insulating synthetic wool fabric made from PET or other synthetic fibers. Fleece traditionally comes in different thickness: micro, 100, 200, and 300, with 300 being the thickest and least flexible. For example, the sleeve may be formed from a fleece material such as a lightweight/microfleece (100 thickness—about 200-240 grams per yard), medium/regular weight (200 thickness—about 320-380 grams per yard), and heavy weight (300 thickness—about 400 and over grams per yard). Desirably, the sleeve is made from a medium weight fleece typically referred to as “blanket weight.” Generally, fleece provides warmth without weight, soft to the touch, does not shrink, ravel or run, is water-resistant, and has moisture wicking properties.

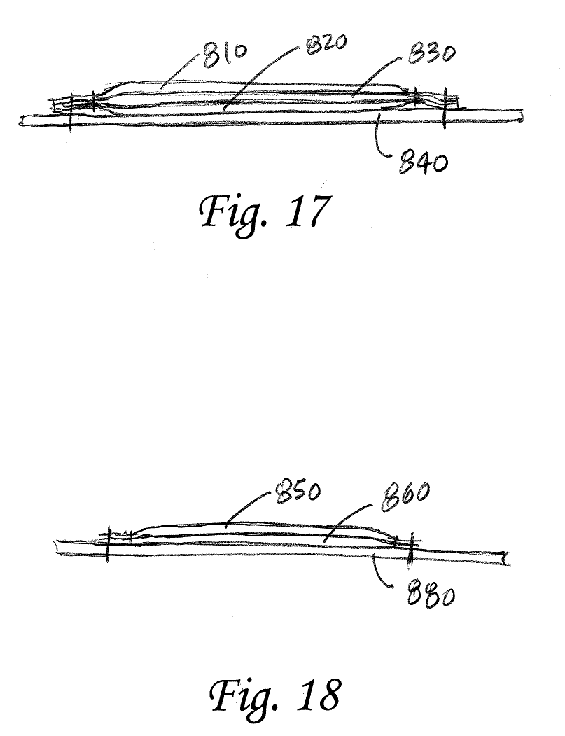

FIG. 17 illustrates one embodiment of the elbow portion of the various devices in accordance with the present invention in which the elbow portion may be formed from a three-piece assembly by first sewing two outer resilient material layers 810 and 820 around a resilient inner layer 830 such as a foam layer. Thereafter, the peripheral edges of the assembly may be sewn to a sleeve 840.

FIG. 18 illustrates another embodiment of the elbow portion of the various devices in accordance with the present invention in which the elbow portion may be formed from a two piece assembly by first sewing an outer resilient material layers 850 to a resilient inner layer 860. Thereafter, the peripheral edge of the outer resilient material layer may be sewn to a sleeve 880.

The sleeve may be contoured by one or more layers of material being cut and sewn together. The sleeve may also comprise an outer decorative design. For example, the devices may include a solid color, patterns of different colors, names, logos, advertising, or other decorative elements. In addition, the design may be disposed on the sleeve and/or pocket portion. The sleeve is generally thinner than the resilient means desirably allowing the poker player to readily bend their arm while wearing the sleeve. The sleeve may be a tubular sleeve.

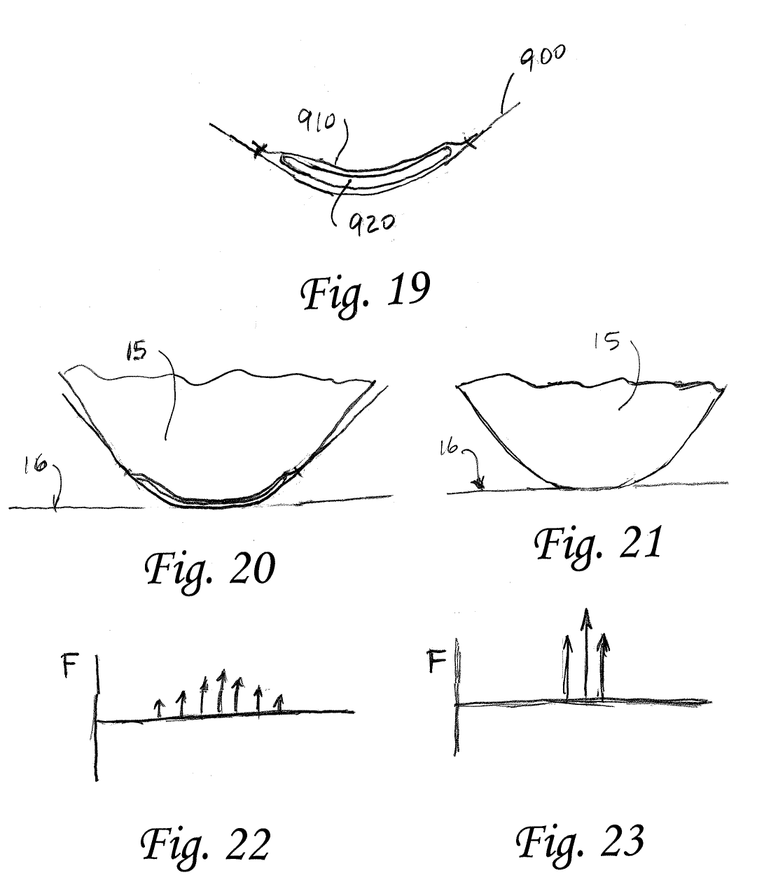

The sleeve may include two layers wherein the two layers are sewn together around the resilient pad. The two layers may also be sewn around the upper and lower ends, which define the opening of the sleeve for inserting the poker player's arm. Alternatively, the sleeve may comprise a first layer, which extends from the poker player's upper arm to the poker player's lower arm. A second layer sized larger than the resilient pad may be employed and sew around the resilient pad to the first layer. For example, FIG. 19 illustrates a cross-section of another embodiment of the elbow portion of the devices, which may include a sleeve layer 900, which extends from the upper arm to the lower arm, and an outer layer 910 which may be sewn and attachable to sleeve layer 900 around a resilient means 920 as described below. The sleeve may be sized to be worn on top of the bare skin of the arm or sized to be worn over a sleeve of a poker player's shirt or other clothing. Also, the elbow sleeve protector may be sized from small, medium, or large, as well as different colors or fabric designs. For example, the length of the various devices may be about 8 inches to about 10 inches, and about 4 inches wide to about 7 inches wide.

Resilient means of the various embodiments may be a resilient pad positioned and disposed in a sealed pocket or unsealed pocket disposable over the elbow of the poker player to provide a cushion to the elbow when the device is worn and the player's elbow is placed on a poker table. The resilient pad may have a length dimension and width dimensions of about 2 inches to about 4 inches. For example, the resilient pad may be rectangular, circular, oval, or other suitable shapes and sizes. The thickness of the pad may be greater than the thickness of the sleeve. For example, the thickness of the pad may be about ⅛-inch or may be greater than about ⅛-inch. The resilient pad may be thicker in the middle and taper toward the edges. The resilient pad may be formed from a fabric or like material such as a cotton/polyester blend, polymer, foam, a rubber, or rubber like material, and/or an elastomeric material, or other suitable material and combinations thereof. The resilient pad may be freely supported in the sealed or unsealed pocket, or the peripheral edges of the resilient pad may be sewn together with an outer layer which are attached to the sleeve. The resilient pad may also contain a fluid such as a gas (e.g., air), or liquid such as a gel material. For example, such a resilient pad may include a fluid or gel material surrounded, encased, or encapsulated in a sealed outer impermeable enclosure such as a sealed plastic or polymeric casing or other impermeable resilient material. The pad is generally kept at room temperature, and after being worn, generally at the temperature of the poker player. In addition, the pad may be a thin hot pack or a cold pack for heating or cooling the poker player's elbow.

As shown in FIG. 20, during a plurality of poker games, a portion of the resilient pad, for example, as shown in FIG. 19, is compressed to a thickness less than the initial thickness between a surface of a poker table 16 and a poker player's elbow 15 of the arm with a forearm of the arm elevated above the poker table to cushion and support the weight of the poker player's arm. In a similar manner, during a plurality of poker games, a portion of the resilient pad, for example, the resilient pad portion as shown in FIGS. 17 and 18, is compressed to a thickness less than the initial thickness between a surface of a poker table 16 and a poker player's elbow 15 of the arm with a forearm of the arm elevated above the poker table to cushion and support the weight of the poker player's arm. In addition, where a thick soft sleeve material such as a medium or heavy weight fleece, the sleeve will also aid in cushioning and supporting the elbow and distributing the weight of the poker player's arm on the table. FIG. 21 illustrates a poker player's elbow 15 directly on a table 16 during a plurality of poker games.

FIG. 22 illustrates the distribution of forces exerted on the poker player's elbow when wearing the devices of the present invention when playing poker at a poker table, e.g., as shown in FIG. 20. FIG. 23 illustrates the forces exerted on the poker player's elbow when not wearing the devices of the present invention when playing poker at a poker table, e.g., as shown in FIG. 21. As observed in comparing FIGS. 22 and 23, the forces exerted on the lowermost portion of the player's elbow during play of a poker game is reduced compared to the forces exerted on the lowermost portion of the player's elbow for supporting the player's elbow directly on the poker table. In addition, as shown in FIG. 22, the devices of the present invention result in the pad distributing the weight of the player's arm on the elbow over a greater area (only one dimension being shown in FIG. 22 compared to the area (only one dimension being shown in FIG. 23) for distributing the weight of the player's arm on the elbow directly disposed on the poker table. Further, as best shown in FIG. 20, during a plurality of poker games, a portion of the resilient pad is compressed to a thickness less than the initial thickness (e.g., as shown in FIG. 19) between a surface of a poker table 16 and the poker player's elbow 15 with a forearm of the arm elevated above the poker table to support the weight of the poker player's arm. Further, the resilient pad also cushions the elbow of the poker player when engaging the poker table.

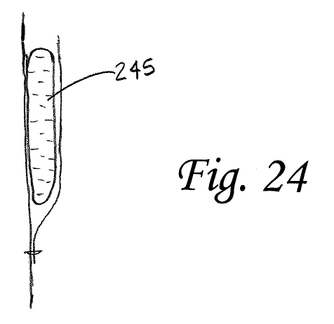

As shown in FIG. 24, the removable hot pack and cold pack may be held in an open pocket attached to the sleeve. The pocket may be disposed away from and spaced-apart from the resilient means covering the poker player's elbow. For example, a second layer of material may be sized larger than the hot pack and cold pack sewn around the peripherally-extending edges to define an opening for inserting and receiving the hot pack and cold pack. The open pocket may be square or rectangular shaped, and sewn on three of the four edges. The pocket may be sized to be about 3 inches by about 4 inches, as well as other sizes. The removable hot pack and cold pack may be hot and cold packs which are chemically activated or heated in a microwave or freezer. The hot or cold packs may provide therapeutic treatment to the arm during play of a poker tournament. The application of hot and cold treatment may be similar to hot and cold treatment for treating back. The hot packs typically last approximately eight hours and the cold packs typically last one to two hours. Replacement packs may also be provided. The first open pocket may be disposed 2 inches to 3 inches above the elbow pad to allow and not obstruct the ability of the poker player to bend their arm. The gel pack may be about 3 inches by 4 inches. The hot pack and cold pack may be disposed on the outside of the poker player's upper arm between, for example, the biceps brachii muscle and the triceps brachii muscle. It will be appreciated that the hot pack and cold pack may be placed in other locations along the arm of the poker player. For example, the pocket for the hot or cold pack may be placed on the back of the poker player's arm above the elbow. The hot and cold packs may be commercially available gel packs which includes a gel which retains a cold or hot temperature, and remains pliable after being subjected to freezing temperatures or microwave radiation in a conventional microwave oven for a sufficient period of time. In addition, other types of heating and cooling means may be suitably employed. For example, electrical heating and cooling means may be employed. In addition, a plurality of pockets may be attached to the sleeve for holding a plurality of hot or cold packs.

The second open pocket may be disposed above the first open pocket along the outside of the upper arm and sized for receiving an electronic device, a cell phone or MP3 player, or other items. For example, the second pocket may be sewn onto the sleeve on three sides. The first and second pockets may extend over and overlap the resilient pad portions of the devices. In the embodiments, the pocket for holding the hot or cold packs may extend horizontally or vertically along the back of the poker player's arm and the pocket for holding the various items may extend vertically or horizontally along the front of the poker player's arm. Other possible placements of the pockets may be possible.

The straps may extend around the forearm and the upper portion or bicep of the arm to minimize any up and down movement on the device on the player's arm. While two straps are shown in the drawings, it will be appreciated that only one may be needed for maintaining the sleeve on the poker player's arm, e.g., reduce the likelihood of the sleeve sliding relative to the poker player's arm. The strap may be a belt material, VELCRO hook-and-loop straps, elastic bands, strings, or ties. In addition, the straps need not extend around the entire arm of the poker player. For example, as shown in FIG. 4, the strap may comprise a first portion 182 and a second portion 184. An end 183 of first portion 182 may be attached to sleeve 122 and an end 185 may be attached to sleeve 122. For example, where the portions 182 and 184 are hook-and-loop fasteners, the free end may be readily connected together to hold the sleeve on the arm of the poker player. Where two pockets are provided, the strap may be disposed around the sleeve between first open pocket and second open pocket. In other embodiments, the sleeve may include fasteners such as hook-and-loop fasteners, snaps, buttons, zippers, etc. which allow, for example, the sleeve to be wrapped around the poker player's arm and the overlapping longitudinal edges of the sleeve may be releasably attached together.

An antibacterial material may be sprayed on or impregnated into the entire sleeve or a portion of the sleeve, such as on and around the elbow portion of the sleeve.

In preferred embodiments, the sleeve may be formed from two generally rectangular or tapering layers of medium weight fleece, for example as shown in FIGS. 4, 8, 12, and 16. A resilient oval-shaped pad is sewn to the inside of the sleeve may comprise a foam layer sandwiched between two layers of felt or felt-like material. The pockets may comprise a generally square or rectangular layer of medium weight fleece which is attached to the outside of the sleeve. Such a device is comfortable when worn on a bare arm, as well as retaining heat from escaping from the surface of the arm covered by the sleeve.

Desirably, the devices for reducing elbow and arm fatigue of a poker player during play of a plurality of poker games may be fabricated from material which allow the devices to be washable between uses. The various materials forming the devices may also be resilient, not include any rigid or hard materials or panels, and provide a smooth outer surface and not a rough outer surface allowing the sleeve to easily and generally with little or no friction slide on the poker table. Further, the sleeve may encircle the poker player's arm. However, the sleeve need not extend entirely around the poker player's arm, for example, the sleeve may include one or more cutouts or openings such as along the inside of the elbow or other portions of the arm, or only extend along a portion of the poker player's arm such as the bottom portion of the arm and held in place by the straps. The sleeve may be snug fit or loosely fit over the poker player's arm. The sleeve may extend along the top of the inside of the poker player's elbow and desirably does not include padding on the inside portion of the poker player's elbow.

In another aspect of the present invention, a kit may be provided for reducing elbow and arm fatigue of a poker player during play of a plurality of poker games. Such a kit may include at least one sleeve as described above and several hot and/or cold packs. The sleeves may be contoured and configured, with one for wearing on the right arm, and one for wearing on the left arm.

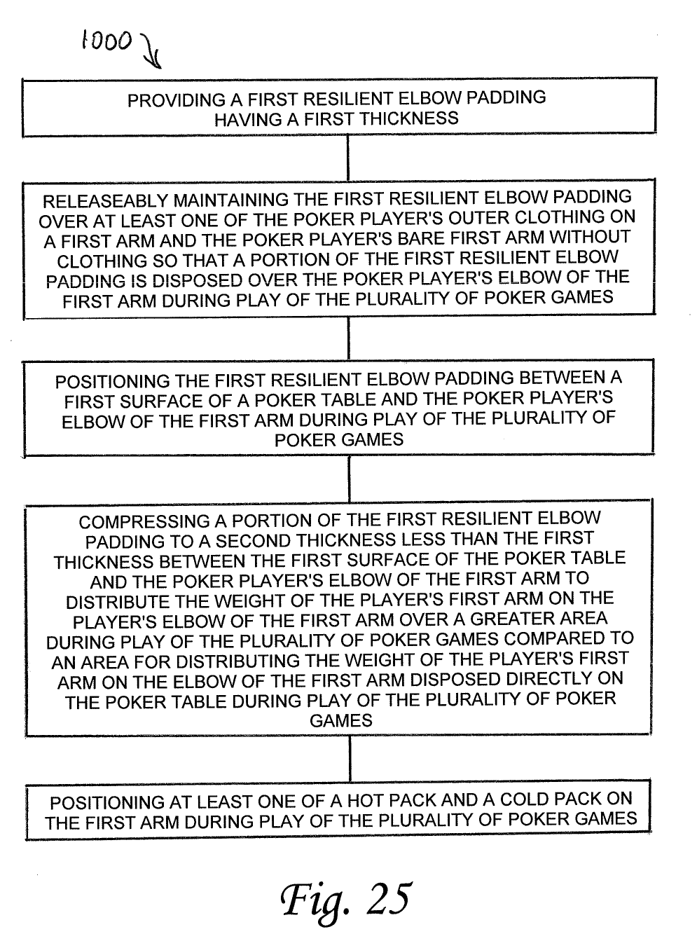

In another aspect of the present invention, a method is provided for reducing elbow and arm fatigue for poker players when playing a poker game. With reference to FIG. 25, a method 1000 is provided for reducing elbow and arm fatigue of a poker player during play of a poker game. Method 1000 includes providing a first elbow pad having a length dimension and a width dimension of about 2 inches to about 4 inches, and a first thickness, positioning the first elbow pad between a first surface of a poker table and the poker player's elbow of a first arm, compressing a portion of the first elbow pad to a second thickness less than the first thickness between the first surface of the poker table and the poker player's first elbow of the first arm with a forearm of the first arm elevated above the poker table to distribute the weight of the poker player's first arm on the poker player's first elbow over a greater area during play of the plurality of poker games compared to an area for distributing the weight of the poker player's first arm on the first elbow disposed directly on the poker table during play of the plurality of poker games, and positioning at least one of a hot pack and a cold pack on the first arm during play of the plurality of poker games.

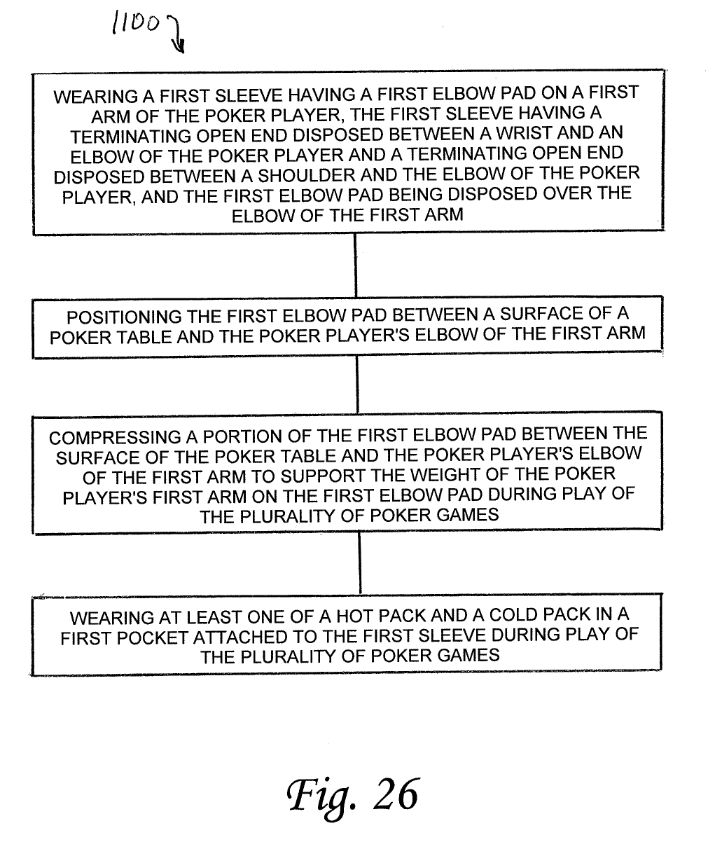

FIG. 26 illustrates another method 1100 for reducing elbow and arm fatigue of a poker player during play of a poker game. Method 1100 includes wearing a first resilient sleeve having a first resilient elbow pad on a first arm of the poker player, the first sleeve having an open end disposed between the wrist and an elbow and an open end disposed between a shoulder and the elbow, and the first elbow pad being disposed over the elbow of the first arm, positioning the elbow pad between a surface of a poker table and the poker player's elbow of the first arm, compressing a portion of the first resilient elbow pad between a surface of the poker table and the poker player's first elbow of the first arm with a forearm of the first arm elevated above the poker table to resiliently support the weight of the poker player's first arm on the first resilient elbow pad during play of the plurality of poker games, and wearing at least one of a hot pack and a cold pack in a first pocket attached to the first sleeve during play of the plurality of poker games.

Although the invention has been particularly shown and described with reference to certain preferred embodiments, it will be readily appreciated by those of ordinary skill in the art that various changes and modifications may be made therein, without departing from the spirit and scope of the invention.

1. A method for reducing elbow and arm fatigue of a poker player during play of a plurality of poker games, the method comprising:
providing a first resilient elbow padding having a first thickness;
releaseably maintaining the first resilient elbow padding over at least one of the poker player's outer clothing on a first arm and the poker player's bare first arm without clothing so that a portion of the first resilient elbow padding is disposed over the poker player's elbow of the first arm during play of the plurality of poker games;
positioning the first resilient elbow padding between a first surface of a poker table and the poker player's elbow of the first arm during play of the plurality of poker games; and
compressing a portion of the first resilient elbow padding to a second thickness less than the first thickness between the first surface of the poker table and the poker player's elbow of the first arm to distribute the weight of the poker player's first arm on the poker player's elbow of the first arm over a greater area during play of the plurality of poker games compared to an area for distributing the weight of the poker player's first arm on the elbow of the first arm disposed directly on the poker table during play of the plurality of poker games.

2. The method of claim 1 further comprising positioning at least one of a hot pack and a cold pack on the first arm during play of the plurality of poker games.

3. The method of claim 1 further comprising alternating positioning at least one hot pack and at least one cold pack on the first arm during play of the plurality of poker games.

4. The method of claim 1 wherein the releaseably maintaining the first resilient elbow padding comprises releasably maintaining the first resilient elbow padding asymmetrically disposed over the poker player's elbow of the first arm.

5. The method of claim 1 wherein the releaseably maintaining comprises releasably maintaining about ⅓ of the first resilient elbow padding over the poker player's elbow of the first arm, and about ⅔ of the first resilient elbow padding over the poker player's forearm of the first arm.

6. A method for reducing elbow and arm fatigue of a poker player during play of a plurality of poker games, the method comprising:
providing a first resilient elbow padding having a first thickness;
releaseably maintaining the first resilient elbow padding over at least one of the poker player's outer clothing on a first arm and the poker player's bare first arm without clothing so that a portion of the first resilient elbow padding is disposed over the poker player's elbow of the first arm during play of the plurality of poker games;
positioning the first resilient elbow padding between a first surface of a poker table and the poker player's elbow of the first arm during play of the plurality of poker games;
compressing a portion of the first resilient elbow padding to a second thickness less than the first thickness between the first surface of the poker table and the poker player's elbow of the first arm to distribute the weight of the poker player's first arm on the poker player's elbow of the first arm over a greater area during play of the plurality of poker games compared to an area for distributing the weight of the poker player's first arm on the elbow of the first arm disposed directly on the poker table during play of the plurality of poker games;
providing a second resilient elbow padding having a third thickness;
releaseably maintaining the second resilient elbow padding over at least one of the poker player's outer clothing on a second arm and the poker player's bare second arm without clothing so that a portion of the second resilient elbow padding is disposed over the poker player's elbow of the second arm during play of the plurality of poker games;
positioning the second resilient elbow padding between a second surface of the poker table and the poker player's elbow of the second arm during play of the plurality of poker games; and
compressing a portion of the second resilient elbow padding to a fourth thickness less than the third thickness between the second surface of the poker table and the poker player's elbow of the second arm to distribute the weight of the poker player's second arm on the poker player's elbow of the second arm over a greater area during play of the plurality of poker games compared to an area for distributing the weight of the poker player's second arm on the elbow of the second arm disposed directly on the poker table during play of the plurality of poker games.

7. The method of claim 6 further comprising positioning at least one of a hot pack and a cold pack on the first arm during play of the plurality of poker games, and positioning at least one of a hot pack and a cold pack on the second arm during play of the plurality of poker games.

8. The method of claim 6 further comprising alternating positioning at least one hot pack and at least one cold pack on the first arm during play of the plurality of poker games, and alternating positioning at least one hot pack and at least one cold pack on the second arm during play of the plurality of poker games.

9. The method of claim 6 wherein the releaseably maintaining the first resilient elbow padding comprises releasably maintaining the first resilient elbow padding asymmetrically disposed over the poker player's elbow of the first arm, and the releaseably maintaining the second resilient elbow padding comprises releasably maintaining the second resilient elbow padding asymmetrically disposed over the poker player's elbow of the second arm.

10. The method of claim 6 wherein the releaseably maintaining the first resilient elbow padding comprises releasably maintaining about ⅓ of the first resilient elbow padding over the poker player's first elbow of the first arm, and about ⅔ of the first resilient elbow padding over the poker player's forearm of the first arm, and the releaseably maintaining the second resilient elbow padding comprises releasably maintaining about ⅓ of the second resilient elbow padding over the poker player's elbow of the second arm, and about ⅔ of the second resilient elbow padding over the poker player's forearm of the second arm.

11. A method for reducing elbow and arm fatigue of a poker player during play of a plurality of poker games, the method comprising:
wearing a first sleeve having a first elbow pad on a first arm of the poker player, the first sleeve having a terminating open end disposed between a wrist and an elbow of the poker player and a terminating open end disposed between a shoulder and the elbow of the poker player, and the first elbow pad being disposed over the elbow of the first arm;
positioning the first elbow pad between a surface of a poker table and the poker player's elbow of the first arm; and
compressing a portion of the first elbow pad between the surface of the poker table and the poker player's elbow of the first arm to support the weight of the poker player's first arm on the first elbow pad during play of the plurality of poker games.

12. The method of claim 11 further comprising wearing at least one of a hot pack and a cold pack in a first pocket attached to the first sleeve during play of the plurality of poker games.

13. The method of claim 11 further comprising alternating wearing at least one of a hot pack and at least one of a cold pack in a first pocket attached to the first sleeve during play of the plurality of poker games.

14. The method of claim 11 further comprising holding at least one of an electronic device, keys, and glasses in a second pocket attached to the first sleeve during play of the plurality of poker games.

15. The method of claim 11 wherein the first elbow pad comprises at least one fabric layer and a resilient foam layer.

16. The method of claim 11 wherein the first elbow pad comprises a fluid.

17. The method of claim 11 wherein the first sleeve loosely fits over the first arm of the poker player.

18. The method of claim 11 wherein the first sleeve comprises a blanket weight fleece material.

19. The method of claim 11 wherein the first sleeve comprises at least one outer decorative design.

20. The method of claim 11 wherein the wearing the first sleeve comprises wearing the first sleeve so that the first elbow pad is asymmetrically disposed over the poker player's elbow of the first arm.

21. The method of claim 11 wherein the wearing the first sleeve comprises wearing the first sleeve so that about ⅓ of the first elbow pad is disposed over the poker player's elbow of the first arm, and about ⅔ of the first elbow pad is disposed over the poker player's forearm of the first arm.

22. A method for reducing elbow and arm fatigue of a poker player during play of a plurality of poker games, the method comprising:
wearing a first sleeve having a first elbow pad on a first arm of the poker player, the first sleeve having a terminating open end disposed between the wrist and an elbow of the poker player and a terminating open end disposed between a shoulder and the elbow of the poker player, and the first elbow pad being disposed over the elbow of the first arm;
positioning the first elbow pad between a first surface of a poker table and the poker player's elbow of the first arm;
compressing a portion of the first elbow pad between the first surface of the poker table and the poker player's elbow of the first arm to support the weight of the poker player's first arm on the first elbow pad during play of the plurality of poker games;
wearing a second sleeve having a second elbow pad on a second arm of the poker player, the second sleeve having a terminating open end disposed between the wrist and an elbow of the poker player and a terminating open end disposed between a shoulder and the elbow of the poker player, and the second elbow pad being disposed over the elbow of the second arm;
positioning the second elbow pad between a second surface of the poker table and the poker player's elbow of the second arm; and
compressing a portion of the second elbow pad between the second surface of the poker table and the poker player's elbow of the second arm to support the weight of the poker player's second arm on the second elbow pad during play of the plurality of poker games.

23. The method of claim 22 further comprising wearing at least one of a hot pack and a cold pack in a first pocket attached to the first sleeve during play of the plurality of poker games, and wearing at least one of a hot and a cold pack in a second pocket attached to the second sleeve during play of the plurality of poker games.

24. The method of claim 22 and further comprising alternating wearing at least one of a hot pack and at least one of a cold pack in a first pocket attached to the first sleeve during play of the plurality of poker games, and alternating wearing at least one of a hot pack and at least one of a cold pack in a second pocket attached to the second sleeve during play of the plurality of poker games.

25. The method of claim 22 further comprising holding at least one of an electronic device, keys, and glasses in a pocket attached to at least one of the first sleeve and the second sleeve during play of the plurality of poker games.

26. A device for reducing elbow and arm fatigue of a poker player during play of a plurality of poker games, said device comprising:
means for releasably covering a portion of at least one of the poker player's outer clothing on a first arm and the poker player's bare first arm without clothing so that a portion of the covering means is disposed over the poker player's elbow of the first arm during play of the plurality of poker games, said covering means having an upper terminating end positionable below the shoulder of the poker player and a lower terminating end positionable above the wrist of the poker player; and
means, attached to said covering means and positionable over the elbow of the first arm of the poker player, for distributing the weight of the poker player's first arm on the poker player's first elbow over a greater area of the poker table during play of the plurality of poker games compared to an area for distributing the weight of the poker player's first arm on the first elbow disposed directly on the poker table during play of the plurality of poker games.

27. The device of claim 26 further comprising means, attached to said covering means and positionable over a first portion of the poker player's first arm, for at least one of heating and cooling a portion of the poker player's first arm during play of the plurality of poker games.

28. The device of claim 26 further comprising means, attached to said covering means and positionable over a second portion of the poker player's first arm, for holding at least one of an electronic device, keys, and glasses during play of the poker game.

29. The device of claim 26 wherein said means for distributing is asymmetrically disposable over the poker player's elbow of the first arm.

30. The device of claim 26 wherein said means for distributing comprises about ⅓ of said means for distributing disposable over the poker player's elbow of the first arm, and about ⅔ of said means for distributing disposable over the poker player's forearm of the first arm.

31. The device of claim 26 wherein the covering means comprises a blanket weight fleece material.

32. The device of claim 26 wherein the covering means comprises at least two layers medium/regular weight fleece material weighing about 320-380 grams per yard sewn together along the peripheral longitudinally-extending edges, and said means for distributing being disposed between said peripheral longitudinally-extending edges.

33. The device of claim 26 wherein the means for distributing comprises a fluid.

34. The device of claim 26 wherein the covering means loosely fits over the first arm of the poker player.

35. The device of claim 26 wherein the covering means comprises an outer decorative design.

36. A kit for reducing elbow and arm fatigue of a poker player during play of a plurality of poker games, said kit comprising:
a plurality of devices of claim 26, a first one of the plurality of devices positionable on a right arm of the poker player, and a second one of the plurality of devices positionable on a left arm of the poker player.

37. A kit for reducing elbow and arm fatigue of a poker player during play of a plurality of poker games, said kit comprising:
a plurality of devices of claim 27, a first one of the plurality of devices positionable on a right arm of the poker player, and a second one of the plurality of devices positionable on a left arm of the poker player.

38. A method for reducing elbow and arm fatigue of a poker player during play of a plurality of poker games, the method comprising:
inserting a first arm of the poker player through the covering means of the first device of claim 26; and
compressing the means for distributing between a surface of a poker table and the poker player's first elbow of the first arm to resiliently support the weight of the poker player's first arm during play of the plurality of poker games.

39. A method for reducing elbow and arm fatigue of a poker player during play of a plurality of poker games, the method comprising:
inserting a first arm of the poker player through the covering means of the first device of claim 27;
compressing the means for distributing between a surface of a poker table and the poker player's first elbow of the first arm to resiliently support the weight of the poker player's first arm during play of the plurality of poker games; and
using the means for at least one of heating and cooling to at least one of heat and cool a portion of the poker player's first arm during play of the plurality of poker games.

40. The method of claim 39 further comprising using the means for at least one of heating and cooling comprises alternating heating and cooling the portion of the first arm of the poker player during play of the plurality of poker games.

41. A method for reducing elbow and arm fatigue of a poker player during play of a plurality of poker games, the method comprising:
providing a plurality of devices of claim 26;
inserting a first arm of the poker player through the covering means of a first one of the plurality of devices;
inserting a second arm of the poker player through the covering means of a second one of the plurality of devices;
compressing the means for distributing of the first device between a first surface of a poker table and the poker player's elbow of the first arm to resiliently support the weight of the poker player's first arm during play of the plurality of poker games; and
compressing the means for distributing of the second device between a second surface of the poker table and the poker player's elbow of second arm to resiliently support the weight of the poker player's second arm during play of the plurality of poker games.

42. A method for reducing elbow and arm fatigue of a poker player during play of a poker game, the method comprising:
providing a plurality of devices of claim 27;
inserting a first arm of the poker player through the covering means of a first one of the plurality of devices;
inserting a second arm of the poker player through the covering means of a second one of the plurality of devices;
compressing the means for distributing of the first device between a first surface of a poker table and the poker player's elbow of the first arm to resiliently support the weight of the poker player's first arm during play of a poker game;
compressing the means for distributing of the second device between a second surface of the poker table and the poker player's elbow of the first arm to resiliently support the weight of the poker player's first arm during play of a poker game;
using the means for at least one of heating and cooling to at least one of heat and cool a portion of the poker player's first arm during play of a plurality of poker games; and
using the means for at least one of heating and cooling to at least one of heat and cool a portion of the poker player's second arm during play of a plurality of poker games.

43. The method of claim 42 further comprising using the means for at least one of heating and cooling to alternate heating and cooling the portion of the first arm of the poker player during play of the plurality of poker games, and further comprising using the means for at least one of heating and cooling to alternate heating and cooling the portion of the second arm of the poker player during play of the plurality of poker games.
